(19) United States
(12) Patent Application Publication
(10) Pub. No.: US 2012/0004684 A1
(43) Pub. Date: Jan. 5, 2012

(54) MEDICAL INSTRUMENT

(76) Inventor: Josef Reinauer, Inzigkofen (DE)

(21) Appl. No.: 12/827,839

(22) Filed: Jun. 30, 2010

(30) Foreign Application Priority Data
Jun. 30, 2010 (DE) 10 2009 031 262.5

(51) Int. Cl. A61B 17/00

(52) U.S. Cl. 606/205

(57) ABSTRACT

A medical instrument with a shaft having a tool on a distal end including a movable jaw member and a handle on a proximal end with a movable gripping member, wherein the jaw and gripping members are in operational connection by a traction/compression element, and wherein a force-limiting device is positioned between the gripping and jaw members for limiting force transmitted from the gripping member onto components in operational connection with the gripping member. Upon exceeding a maximum load, the device reversibly severs the components that are coupled with one another. The force-limiting device includes a housing including two components that can slide with respect to one another with one component mounted rigidly in the housing and the other component positioned to slide axially in the housing against the force of a spring element, such that the two components are coupled by a force transmission element acting on both components.

### CROSS-REFERENCE TO RELATED APPLICATIONS

The present application claims priority of German patent application No. 10 2009 031 262.5 filed on Jun. 30, 2010, the content of which is incorporated herein by reference.

### FIELD OF THE INVENTION

The invention relates to a medical instrument with a shaft having on its distal end a tool that comprises at least one movable jaw member and having on its proximal end a handle equipped with at least one movable gripping member, such that the movable jaw member and the movable gripping member are in operational connection with one another by means of a traction/compression element and such that positioned between the movable gripping member and the movable jaw member is at least one force-limiting device for limiting the force transmitted by force transmission from the movable gripping member onto the components that are in operational connection with the movable gripping member, and when a maximum load is exceeded, said force-limiting device reversibly severs the components that are coupled with one another.

### BACKGROUND OF THE INVENTION

Such medical instruments can include, for instance, grasping, fixation, and dressing forceps, scissors, or similar instruments in which a displaceable tool such as a pivotable jaw member is moved by manual force applied to a displaceable gripping member of the handle.

These known medical instruments, which have applications for instance in endoscopic surgery, comprise a shaft on whose distal end a tool is positioned that comprises at least one movable jaw member and on whose proximal end a handle is positioned that is equipped with at least one movable gripping member. For opening and closing the movable jaw member, the movable jaw member and the movable gripping member are connected with one another by means of a traction/compression element.

The gripping members of the handle, as a rule, are geometrically configured in such a way that the force exerted by the operator's hand on the gripping member is clearly reinforced, in particular in a ratio of 10:1, in the transmission onto the traction/compression element that is coupled with the jaw member, in order to achieve a sufficiently high closing force on the jaw member even without great exertion.

To prevent the exertion of such great forces by the handle onto the jaw members and/or the traction/compression element as can lead to damage or even destruction of individual components, it is common in the art to equip medical instruments with a force-limiting device, which when a maximum load is exceeded, reversibly severs the components that are coupled to one another.

One example of a known practice is to equip the force transmission mechanism with a predetermined breaking point. This type of force limitation ensures a secure protection of the instrument, but has the disadvantage that the instrument is not immediately employable again, because it must be dismantled to repair the predetermined breaking point.

A generic medical instrument with a reversibly working force-limiting device is known, for instance, from DE 197 31 453 C2. In this known medical instrument the force limitation is configured as an elastically deformable part of the traction/compression element that reversibly deforms itself upon exceeding the maximum load.

On this basis, it is the object of the invention to provide a medical instrument with a force-limiting device, which upon reaching the maximum load, ensures a clearly recognizable reduction of the exerted force.

### SUMMARY OF THE INVENTION

This object is achieved, according to the invention, in such a way that the force-limiting device includes a housing in which two components that can be moved in relation to one another are positioned in such a manner that one component is mounted rigidly in the housing and the other component is positioned so that it can move axially in the housing against the force of a spring element, such that the two components are coupled with one another by at least one force transmission element that acts on both components.

The inventive configuration of the force-limiting device with the two components that can move with respect to one another and are preferably of sleeve-like configuration has the advantage that the spring-loaded axially movable component, when the maximum load is exceeded by the at least one force-limiting element, is abruptly pressed away, so that the force exerted by the movable gripping member of the handle abruptly sinks back almost to zero.

According to a first practical embodiment of the invention, it is proposed that the at least one force transmission element by which the tractive force of the movable gripping member of the handle can be transmitted to the two components should be configured as a sphere. This type of force transmission element constitutes a variant that is especially simple to produce and install.

According to an alternative embodiment of the invention, it is proposed that the at least one force transmission element should be configured as a pivot lever.

It is further proposed with the invention that the contact surfaces on which the at least one force transmission element comes in contact with both components should be configured as surfaces slanting away from one another in such a way that the maximum load for triggering the force-limiting device is a trigonometric function of the force of the force transmission element onto the slanted contact surface of the slidable component, such that the force of the force transmission element onto the slanting contact surface of the slidable component is a trigonometric function of the tractive force exerted by the movable gripping member of the handle onto the slanting contact surface of the rigid component.

For positioning the force-limiting device, it is proposed according to a preferred embodiment of the invention that said device should be positioned in the movable gripping member of the handle in the area of the mechanism that transmits force onto the traction/compression element. The positioning of the force-limiting device in the handle is especially advantageous in endoscopic instruments, because in the handle rather than the shafts of these instruments, owing to the slender configuration of the shafts, there is sufficient space available for accommodating such a device.

The transmission of the tractive force of the movable gripping member of the handle onto the at least one force transmission element occurs, when the force transmission device is positioned in the handle, by means of a tie rod mounted on the movable gripping member of the handle.

To adapt the medical instrument to various application purposes as well as to adapt to various tool jaw members, the maximum load that causes the triggering of the force-limiting device can be adjusted by the spring element that acts on the slidable component.

It is further proposed with the invention that the force-limiting device can be triggered by tactile and/or acoustical and/or optical means in order to indicate to the user without any doubt that the admissible maximum load has been exceeded and that the force-limiting device has been triggered.

Finally, it is proposed with an alternative embodiment of the invention that the force-limiting device should be positioned in the area of the traction/compression element and configured for instance as a part of the traction/compression element.

Further traits and advantages of the invention can be seen from the appended illustrations, in which two embodiments of an inventive medical instrument are shown as examples, without restricting the invention to these embodiments.

### BRIEF DESCRIPTION OF THE DRAWINGS

FIG. 1 shows a schematic side view of an inventive medical instrument.

FIG. 2 shows an enlarged and partly cut-out schematic view of the handle of a medical instrument according to FIG. 1, with the force-limiting device in the non-triggered state.

FIG. 3 shows a view of the handle according to FIG. 2, but with the force-limiting device in the triggered state.

FIG. 4 shows a schematic depiction of the force distribution inside the force-limiting device.

FIG. 5 shows a schematic view of an alternative embodiment for configuring the force-transmission element.

### DETAILED DESCRIPTION OF THE INVENTION

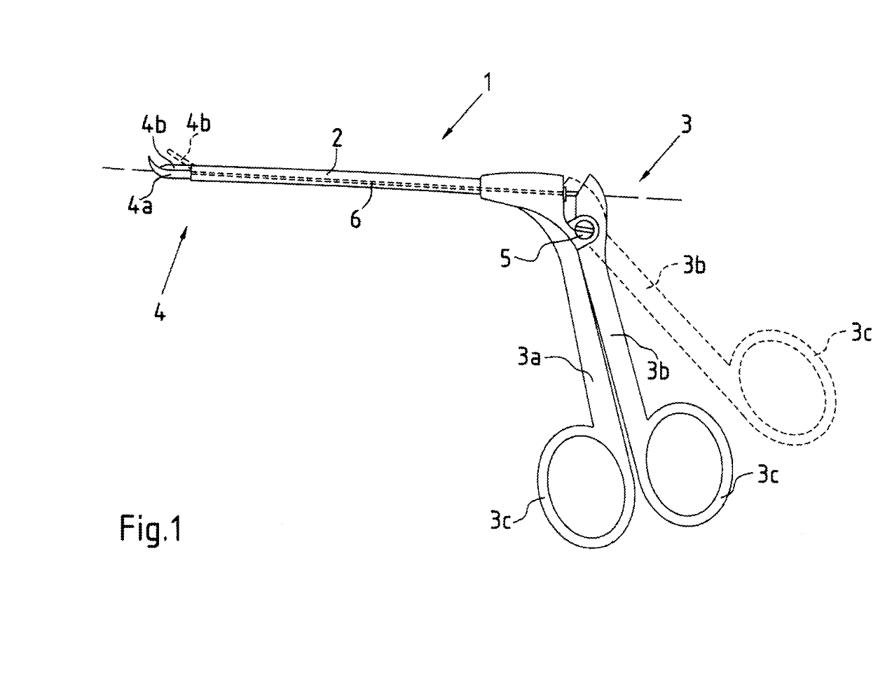

The first illustration, FIG. 1, presents schematically a medical instrument that can take the form, for instance, of a grasping, fixation, or dressing forceps, scissors, or similar instrument in which a displaceable tool such as a pivotable jaw member is moved by hand force.

The medical instrument 1, shown only schematically in the illustration, consists essentially of a hollow shaft 2 on whose proximal end a handle 3 is positioned that consists of a rigid gripping member 3a and a gripping member 3b that can pivot with respect to the rigid gripping member 3a. Positioned on the distal end of the shaft 2 is a tool 4, which consists of a rigid jaw member 4a and jaw member 4b that can pivot with respect to the rigid jaw member 4a. Which of the two gripping members 3a and 3b of the handle 3 is configured so that it can pivot or otherwise be displaced, is irrelevant to the means of functioning of the medical instrument 1.

As can further be seen from FIG. 1, the pivotable jaw member 4b of the tool 4 and the gripping member 3b of the handle 3 that can pivot around a pivot axis 5 are in operational connection with one another by means of a traction/compression element 6, mounted so that it can slide axially in the hollow shaft 2, in such a way that displacing the gripping member 3b of the handle 3 moves the pivotable jaw member 4b of the tool 4 from the closed position (shown with solid line in FIG. 1) into the open position (dotted line in FIG. 1) or vice versa. The relevant position of the pivotable gripping member 3b of the handle 3 in each case is likewise shown with solid line (for closed position) and dotted line (for open position).

As can be seen by juxtaposing FIGS. 1 through 3, the pivotable gripping member 3b is connected by a rocker arm 7 with the axially slidable traction/compression element 6 and the distance from the pivot axis 5 to the position at which the rocker arm 7 is connected with the axially slidable traction/compression element 6 is substantially shorter than the distance from the pivot axis 5 to the finger loop 3c positioned on the outer end of the pivotable gripping member 3b. The transmission ration is approximately 10:1, so that the closing force exerted by the user's hand is magnified tenfold.

To prevent such great forces from being exerted by the handle 3 onto the jaw members 4a and 4b and/or the traction/compression element 6 as could lead to damage or even destruction of individual components, the medical instrument 1 comprises a force-limiting device 8, which when a maximum load is exceeded, reversibly severs the components that are coupled to one another.

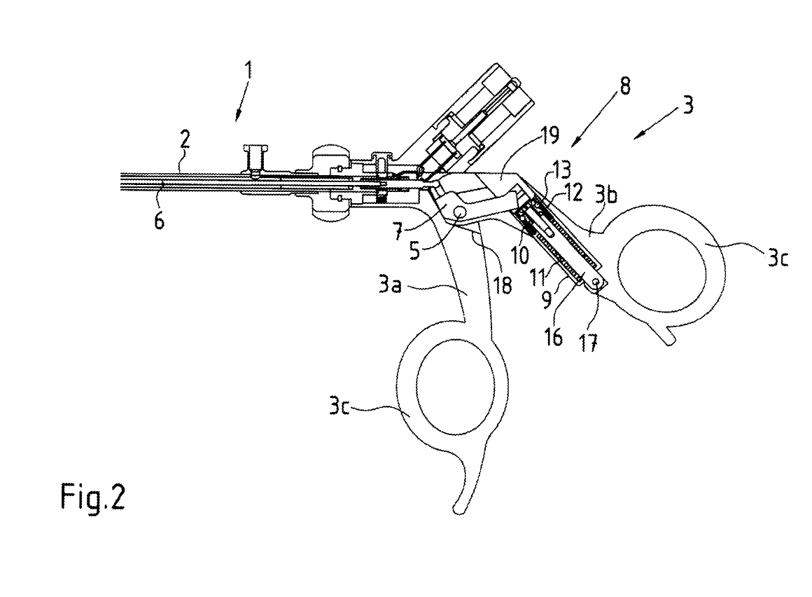

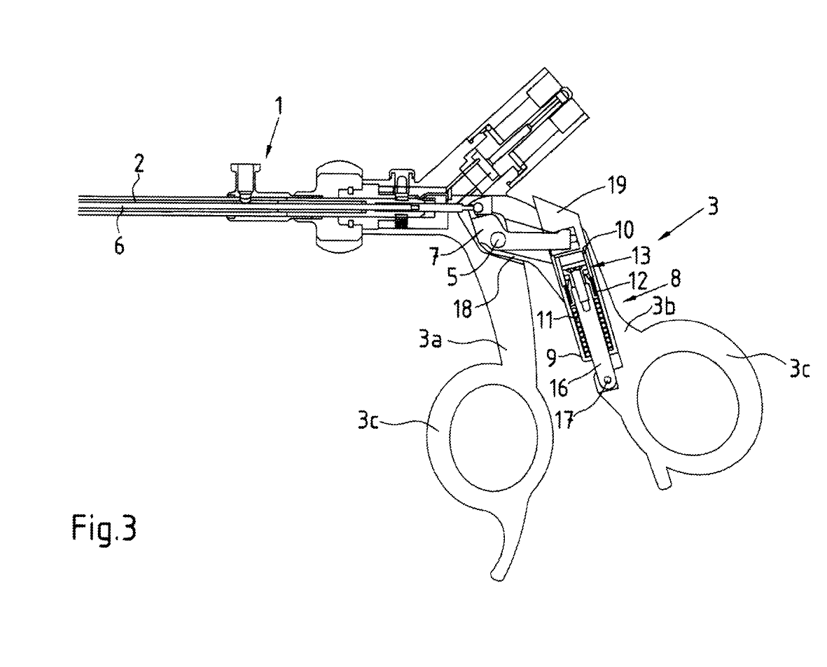

In the illustrated embodiment of the medical instrument 1, the force-limiting device 8, as can be seen from FIGS. 2 and 3, is positioned in the movable gripping member 3b of the handle 3 in the area of the mechanism for transmitting force onto the traction/compression element 6.

Alternatively to the illustrated position of the force-limiting device 8 in the handle 3, it is also possible to position the force-limiting device 8 in the area of the traction/compression element 6 and, for instance, to configure it as part of the traction/compression element 6.

The structure and functioning of the force-limiting device 8 can be seen from FIGS. 2 through 5.

The force-limiting device 8 consists essentially of a housing 9 in which a rigid component 10 and a component 12, which can slide axially against the force of a spring element 11 and is mounted in the housing 9, are mounted so that they can be slid relative to one another and where the two components 10 and 12 are coupled to one another by means of a force transmission element 13 that acts on both components 10 and 12 and transmits the tractive force F_{z} of the displaceable gripping member 3b of the handle 3 onto the two components 10, 12.

As can be seen from FIGS. 2 and 3, the contact surfaces 10a and 12a of the two components 10 and 12, on which the force transmission element 13 is in contact with the two components 10, 12, are configured as surfaces slanting toward one another.

The at least one force transmission element 13, which is in contact with contact surfaces 10a and 12a, is configured as a sphere 14 in the first embodiment shown in FIGS. 2 through 4.

A second, alternative embodiment for configuring the force transmission element 13 is shown in FIG. 5, in which the force transmission element 13 is configured as a pivot arm 15. Other embodiments for configuring the force transmission element 13, such as in the form of a circle segment, are likewise possible.

The transmission of the tractive force F_{z} of the displaceable gripping member 3b of the handle 3 onto the two components 10, 12 by means of the force transmission element 13 occurs in the embodiment shown in FIGS. 2 and 3 by means of a tie rod 16, which is mounted on a pivot point 17 on the displaceable gripping member 3b of the handle 3.

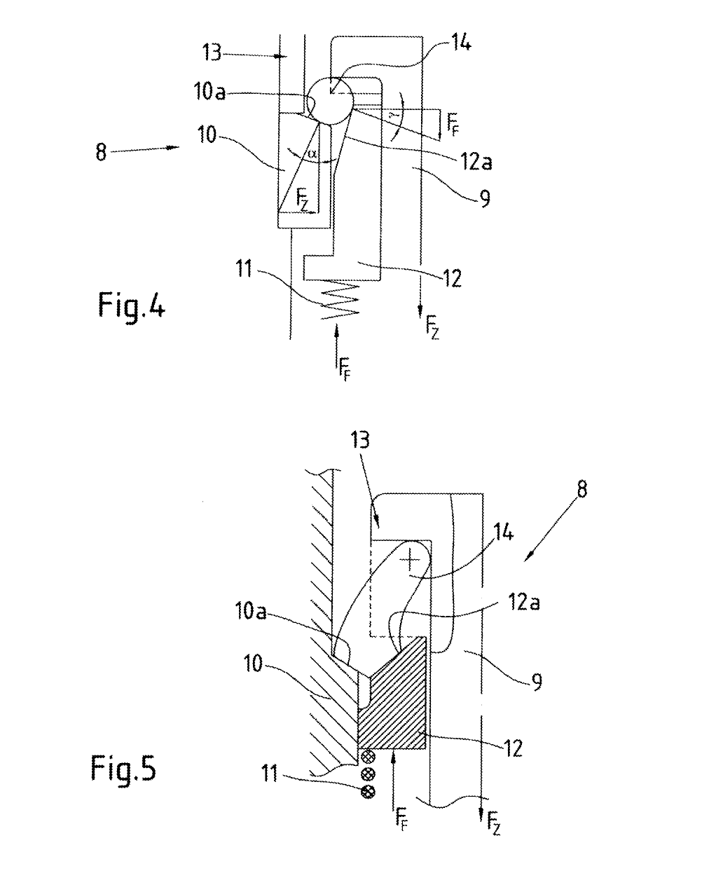

As can be seen from the schematic force diagram in FIG. 4, the maximum load for triggering the force-limiting device 8, which can be adjusted by the spring force F_{f} of the spring element 11, is a trigonometric function of the force of the force transmission element 13 onto the slanting contact surface 12a of the slidable component 12, such that the force of the force transmission element 13 onto the slanting contact surface 12a of the slidable component 12 in turn is a trigonometric function of the maximum force F2 exerted by the displaceable gripping member 3b of the handle 3 onto the slanting contact surface 10a of the rigid component 10.

The illustrated force transmission device 8 operates as follows:

On pivoting the pivotable gripping member 3b of the handle 3 around the pivot axis 5, the tie rod 16 that is jointed to the pivot point 17 and mounted in the housing 9 is moved along with it and drawn downward against the spring force F_{f} of the spring element 11 as a result of the exerted tractive force F_{z}.

As a result of this motion, the rocker lever 7, which is suspended on the upper end of the housing 9 and can pivot around the pivot axis 5, is pivoted and draws the traction/compression element 6 in the proximal direction by means of a form-locked suspension to close the pivotable jaw member 4b of the tool 4.

If at this point the pivotable jaw member 4b cannot be further closed, for instance by gripping an object with the jaw members 4a and 4b of the tool 4, and thus if the traction/compression element 6 can also not be drawn further in the proximal direction, then the tractive force F_{z} on the housing increases because of the mounting of the rocker lever 7 on the housing 9.

After exceeding the maximum load that can be individually adjusted by the spring force F_{f} of the spring element 11, the force-limiting device 8 is abruptly triggered and the tractive force F_{z} sinks almost to zero. In this position the pivotable gripping member 3b of the handle 3 can be further pivoted as far as an end stop 18, but without further actuating the rocker lever 5 and thus the traction/compression element 6.

Responsible for the abrupt triggering of the force-limiting device 8 is the interaction of the two sleeve-like components 10 and 12 with the force transmission element 13, by means of which the tractive force F_{z} is transmitted onto the slanting contact surfaces 10a and 12a of the rigid component 10 as well as of the component 12 that can slide axially against the spring force F_{f}.

The pre-tensed spring 11 presses from below with the spring force F_{f} against the slidable component 12, while the force transmission element 13 presses from above onto the slanting contact surface 12a of the slidable component 12 by means of the tractive force F_{z} generated by the pivotable gripping member 3b and thus generates radial forces. The force transmission element 13 presses simultaneously from above onto the slanting contact surface 10a of the component 10 that is rigidly mounted in the housing 9 and thus likewise generates radial forces. The force transmission element 13 cannot move radially to avoid the pressure because of the positioning in the housing of the force transmission device 8.

Now, however, the tractive force F_{z} applied onto the pivotable gripping member 3b is divided by a trigonometric function into a normal force and a shear force on or along the surface on which the force transmission element 13 is in contact with the component 10, and the resulting shear force in turn into a force perpendicular to the tractive force F_{z}, which in turn acts on the surface of component 11 on which the force transmission element 13 is in contact with the component 11. Here the resulting force on the component 11 is again divided by a trigonometric function into normal force and a shear force on or along the surface on which the force transmission element 13 is in contact with the component 11. This normal force on the surface, on which the force transmission element 13 is in contact with the component 11, is again divided by a trigonometric function, among others, into a force opposed to the spring force F_{f}.

If this force opposed to the spring force F_{f}, which is almost the function of the applied tractive force F_{z}, is greater than the spring force F_{f} that applies the spring element 11 onto the slidable component 12, the slidable component 12 is pressed downward against the force of the spring element 11 so that the force transmission element 13 can move aside radially to the side and come to rest close to the rigid component 10. This raising of the position of the force transmission element 13 on the two components 10 and 12 causes the tie rod 16 to be pulled out in the downward direction out of the housing 9 of the force transmission device 8, so that on the other hand the spring element 11 is pressed together.

Pulling out the tie rod 16 causes an extension of the pivot path of the pivotable gripping member 3b as far as the end stop 18, with simultaneous reduction of the tractive force F_{z} almost to zero.

The resulting spring tensing of the spring element 11 is sufficient to return the slidable component 12 as well as the force transmission element 13 reversibly back into the starting position upon pivoting back the pivotable gripping member 3b into the starting position as in FIG. 2, so that the force-limiting device 8 is once again ready for use.

Because of the abrupt triggering of the force-limiting device 8, the exceeding of the maximum load and the triggering of the force-limiting device 8 is not only perceptible by tactile means to the user but can also be observed acoustically by clearly perceptible impact noise.

In the embodiment shown in FIGS. 2 and 3, the force-limiting device 8 is equipped in addition with a cap 19, which upon triggering the force-limiting device 8, emerges from the pivotable gripping member 3b of the handle 3 in order to alert the user visually as well to the exceeding of the maximum load and the triggering of the force-limiting device 8.

The medical instrument 1 of this configuration is characterized in that the reversibly working force-limiting device 8 causes the tractive force F_{z} abruptly to fall to zero and informs the user without any doubt that the maximum load has been exceeded.

1. A medical instrument with a shaft on whose distal end a tool is positioned comprising at least one movable jaw member and on whose proximal end a handle is positioned that is equipped with at least one movable grip-ping member, such that the movable jaw member and the movable gripping member are in operational connection with one another by means of a traction/compression element and such that between the movable gripping member and the movable jaw member at least one force-limiting device is positioned for limiting the force transmitted in the force transmission from the movable gripping member onto the components that are in operational connection with the movable gripping member and said device, upon exceeding a maximum load, reversibly severs the components that are coupled with one another, wherein
the force-limiting device includes a housing in which two components that can slide in relation to one another are positioned in such a way that one component is mounted rigidly in the housing and the other component is positioned so that it can slide in the housing axially against the force of a spring element, such that both components are coupled with one another by means of at least one force transmission element acting on both components.

2. The medical instrument according to claim 1, wherein a tractive force of the displaceable gripping member of the handle can be transmitted to the two components by means of the at least one force transmission element.

3. The medical instrument according to claim 1, wherein support surfaces, on which the at least one force transmission element is in contact with the two components, are configured as surfaces slanting toward one another.

4. The medical instrument according to claim 1, wherein the at least one force transmission element is configured as a sphere.

5. The medical instrument according to claim 1, wherein the at least one force transmission element is configured as a pivot lever.

6. The medical instrument according to claim 1, wherein the maximum load for triggering the force-limiting device is a trigonometric function of the force of the force transmission element onto the slanting contact surface of the slidable component, such that the force of the force transmission element onto the slanting contact surface of the slidable component is a trigonometric function of the tractive force exerted by the displaceable gripping member of the handle onto the slanting contact surface of the rigid component.

7. The medical instrument according to claim 1, wherein the force-limiting device is positioned in the movable gripping member of the handle in the area of the mechanism for transmitting force onto the traction/compression element.

8. The medical instrument according to claim 1, wherein the maximum load that causes the triggering of the force-limiting device can be adjusted by the spring element acting on the slidable component.

9. The medical instrument according to claim 1, wherein the two components that are coupled with one another by the at least one force transmission element are of sleeve-like configuration.

10. The medical instrument according to claim 1, wherein the at least one force transmission element is coupled with the displaceable gripping member of the handle by a tie rod mounted on the displaceable gripping member of the handle.

11. The medical instrument according to claim 1, wherein the triggering of the force-limiting device can be perceived by tactile and/or acoustical and/or optical means.

12. The medical instrument according to claim 1, wherein upon triggering the force-limiting device, the tractive force applied by the displaceable gripping member of the handle is reduced essentially to zero.

13. The medical instrument according to claim 1, wherein the force-limiting device is positioned in the area of the traction/compression element.
